(19) United States
(12) Patent Application Publication
(10) Pub. No.: US 2022/0130553 A1
(43) Pub. Date: Apr. 28, 2022

(54) METHOD FOR EXTRACTING RELATED DISEASE FROM PATIENT PRESCRIPTION AND PROVIDING EXTRACTED DISEASE RELATED INFORMATION

(71) Applicant: Hyoung-jun Bae, Suwon-si (KR)

(72) Inventor: Hyoung-jun Bae, Suwon-si (KR)

(21) Appl. No.: 17/078,380

(22) Filed: Oct. 23, 2020

(51) Int. Cl. G16H 50/70; G16H 70/60; G16H 15/00; G06Q 30/02; G16H 50/20; G16H 20/10; G16H 10/60; G16H 70/20; G06F 40/295; G16H 70/40; G16H 40/20

(52) U.S. Cl. CPC G16H 50/70; G16H 70/60; G16H 15/00; G06Q 30/0255; G16H 50/20; G16H 40/20; G16H 10/60; G16H 70/20; G06F 40/295; G16H 70/40; G16H 20/10

(57) ABSTRACT

A method for extracting a related disease from a patient prescription and providing extracted disease related information is provided. The method prioritizes a predefined primary disease name based on a medical department and a prescribed drug on a patient prescription, and additionally extracts one or more secondary disease names based on a predefined severity level thereof. In this connection, the disease related information is outputted based on a weight allocated thereto based on whether the extracted disease is the primary disease and based on the disease severity level thereof. Thus, when extracting the related disease based on the drug information on the patient prescription, the primary disease is prevented from being excluded from the output. Further, the disease-related information is output based on the predefined diseases severity level.

### FIELD OF THE INVENTION

The present disclosure relates to a method for extracting a related disease from a patient prescription and providing extracted disease related information.

### BACKGROUND OF THE INVENTION

When a patient receives a drug at a pharmacy based on a prescription as prescribed by a doctor, he/she receives oral or printed descriptions of how to take the drug from a pharmacist. In preparation and sale of the drugs, medication counseling of the drug to be provided to the patient must be performed primarily. The medication counseling may include information including a drug name, a dosage amount, an administration manner, efficacy and effect, a storage method, side effects, measures to be taken when the side effects occur, interactions, and precautions for taking medication.

However, the medication counseling from the pharmacist is based only on patient prescription information, and thus is limited to formulation types, actions, and side effects of the drug, and precautions for taking the drug.

Therefore, importance and necessity of providing more specific and useful information to overcome the patient's disease, such as disease-related information including symptoms and future progression of the disease, nutrients required for the disease treatment and medications taken, and reasons for the requirement, foods to eat and foods to avoid, etc. for example, in patients with high blood pressure or diabetes mellitus have been emerged.

FIG. 1 is a flow chart of a prior art method for extracting a related disease from a patient prescription and providing extracted disease related information. Specifically, the prior art method is disclosed in Korean patent No. 1404503 (title of invention: a method for extracting related disease from patient prescription and providing extracted disease related information).

As shown in FIG. 1, in the method for extracting the related disease from the patient prescription and providing the extracted disease related information using a system that provides disease related information matching the extracted disease name based on the patient prescription, a general related disease name is extracted based on the drug or classification of diseases as recited in the patient prescription (S31). Further, whether at least one of the medical department, whether the medical insurance is applied, and the dosage method is described on the patient prescription may be determined (S32). Then, a specific related disease name is additionally extracted based on at least one of the medical department, whether the medical insurance is applied, and the dosage method (S33-1). Information related to the extracted disease is outputted (S33-2). Weights are allocated to the information related to the extracted disease which in turn are outputted based on the allocated weights (S34). Advertisements related to the disease related information are included into the disease related information (S35).

### SUMMARY OF THE INVENTION

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

A purpose of the present disclosure is to provide a method for extracting a related disease from a patient prescription and providing extracted disease related information, in which exclusion of a primary disease is prevented, and a disease is selected based on a severity level of the disease, and disease-related information about the selected disease is output to a patient to facilitate disease healing and to prevent recurrence thereof and maintain health of the patient.

In one aspect, there is provided a method for extracting a related disease from a patient prescription and providing extracted disease related information using a system, wherein the system includes a patient prescription interface, a controller, a storage, and a disease related information output device, wherein the controller includes a related disease name extraction module, a weighting module, and a disease related information output module, wherein the method comprises: receiving, by the related disease name extraction module, prescription information via the patient prescription interface; extracting, by the related disease name extraction module, at last one related disease name from the prescription information based on a medical department and at least one drug as recited in the prescription information; determining, by the related disease name extraction module, whether at least one predefined primary disease name is present among the at least one extracted disease name; upon determination that the at least one predefined primary disease name is present among the at least one extracted disease name, selecting, by the related disease name extraction module, all of the at least one primary disease name, and further selecting, by the related disease name extraction module, at least one predefined secondary disease name based on a predefined disease severity level thereof; upon determination that the at least one predefined primary disease name is not present among the at least one extracted disease name, selecting, by the related disease name extraction module, at least one predefined secondary disease name based on a predefined disease severity level thereof; allocating, by the weighting module, weights to information related to a disease of each of the at least one selected related disease name, wherein the weights correspond to output orders or priorities of the disease related information when the disease related information output module outputs the disease related information; and outputting, by the disease related information output module, the disease related information based on the weights allocated thereto. In this connection, the weighting module allocates first to third weights to basic information about the same disease most necessary to a patient, first additional depth information about the same disease, and second additional depth information about the same disease having deeper knowledge that that of the first additional depth information, respectively, wherein the first to third weights decrease in this order, wherein the weighting module allocates weights to information about the primary disease and information about the secondary disease, respectively, wherein the weight allocated to the information about the primary disease is higher than the weight allocated to the information about the secondary disease, wherein the weighting module allocates different weights to about information the primary diseases based on disease severity levels thereof, or allocates different weights to information about the secondary diseases based on disease severity levels thereof, wherein a higher weight corresponds to a higher severity level.

In one embodiment, the weighting module performs weighting such that: when first and second diseases have first and second severity levels respectively where the first level is higher than the second level, and three information about the first disease have first higher, medium and lower weights allocated thereto respectively, and three information about the second disease have second higher, medium and lower weights allocated thereto respectively, the second higher weight is higher than the first lower weight.

In one embodiment, the controller further includes a disease information related advertisement module, wherein the method further comprises including, by the disease information related advertisement module, advertisements related to the disease related information into the disease related information to be presented to the patient.

### BRIEF DESCRIPTION OF THE DRAWINGS

FIG. 1 is a flow chart of a prior art method for extracting a related disease from a patient prescription and providing extracted disease related information.

FIG. 2 is a block diagram showing a system for implementing a method for extracting a related disease from a patient prescription and providing extracted disease related information according to an embodiment of the present disclosure.

FIG. 3 is a flow chart of a method for extracting a related disease from a patient prescription and providing extracted disease related information according to an embodiment of the present disclosure.

FIG. 4 is a diagram showing an example of extracting a related disease name based on a medical department in the method according to an embodiment of the present disclosure.

FIG. 5 is a diagram showing a weight corresponding to an output priority or order of information about the extracted related disease based on a disease name and a disease severity in the method according to an embodiment of the present disclosure.

FIG. 6A and FIG. 6B are images showing extracted disease related information and advertisements when a disease extracted from the patient prescription is male menopause and high blood pressure according to an embodiment of the present disclosure.

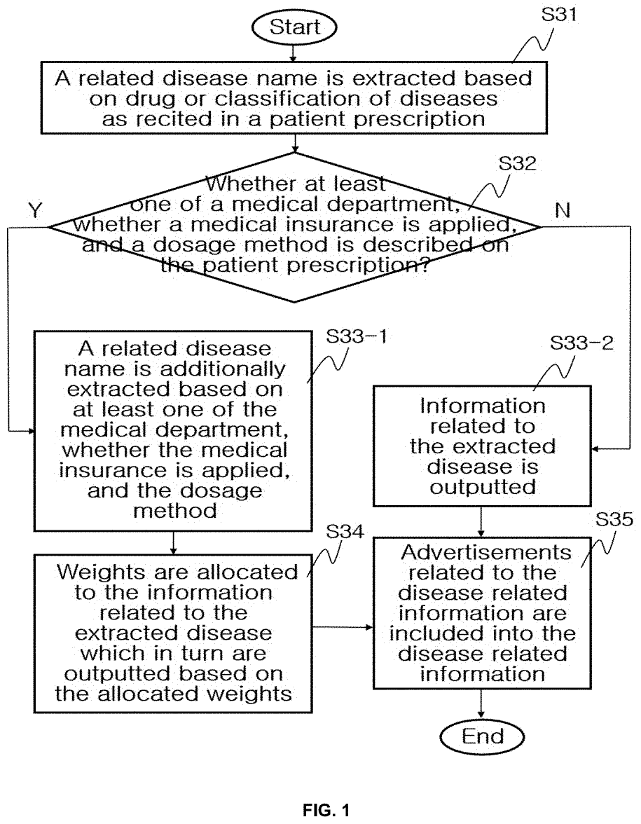

### DETAILED DESCRIPTION OF THE INVENTION

Examples of various embodiments are illustrated and described further below. It will be understood that the description herein is not intended to limit the claims to the specific embodiments described. On the contrary, it is intended to cover alternatives, modifications, and equivalents as may be included within the spirit and scope according to the present disclosure as defined by the appended claims.

Further, descriptions and details of well-known steps and elements are omitted for simplicity of the description. Furthermore, in the following detailed description of the present disclosure, numerous specific details are set forth in order to provide a thorough understanding according to the present disclosure. However, it will be understood that the present disclosure may be practiced without these specific details. In other instances, well-known methods, procedures, components, and circuits have not been described in detail so as not to unnecessarily obscure aspects according to the present disclosure.

The terminology used herein is for the purpose of describing particular embodiments only and is not intended to be limiting according to the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Further, in the present specification, a term such as “unit” or “module” described in the specification mean a unit that processes at least one function or operation, and means that the unit or module may be implemented as one or more hardware or software, or a combination of hardware and software.

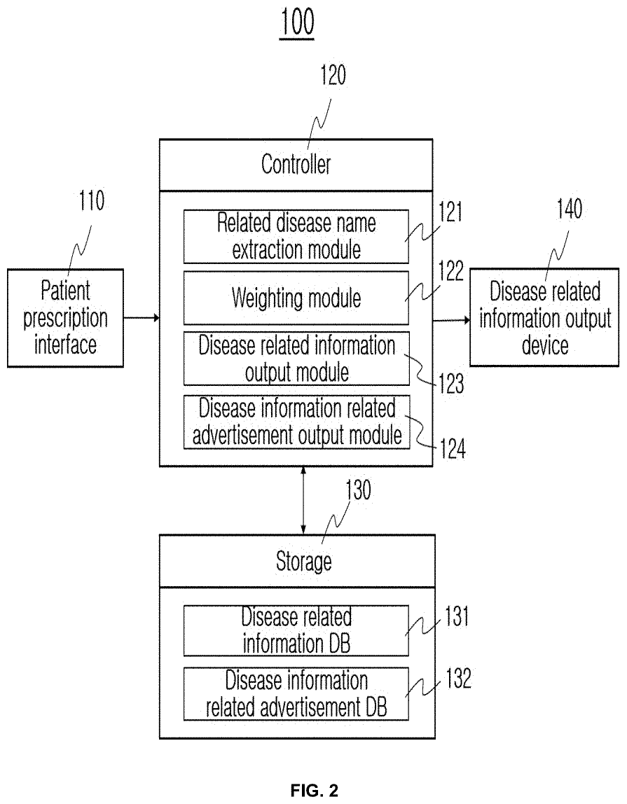

FIG. 2 is a block diagram showing a system for implementing a method for extracting a related disease from a patient prescription and providing extracted disease related information according to an embodiment of the present disclosure.

A system 100 for implementing a method for extracting a related disease from a patient prescription and providing the extracted disease related information according to an embodiment of the present disclosure includes an input device or interface such as a keyboard, a computer, a display, and a printer. The computer stores a program therein for inputting, storing, and outputting various information required to implement the method according to the embodiment of the present disclosure. As shown in FIG. 2, the system may implement functions of a patient prescription interface 110, a controller 120, a storage 130, and a disease related information output device 140.

The patient prescription interface 110 may include a keyboard and an OCR scanner. Thus, the user may use the patient prescription interface 110 to input prescription information to the controller 120 of the computer. The prescription information may include prescribed drugs, classification of diseases, medical departments, whether medical insurance is applied, dosage, taking methods, etc. In some cases, the prescription information may be transmitted and inputted to the controller 120 of the computer from a hospital sever or computer through the Internet or via a wire or in a wireless manner.

The controller 120 may receive the prescription information input via the patient prescription interface 110 and extract a related disease name from the prescription information. The controller 120 may search disease related information database (DB) 131 as described later in the storage 130 for disease related information matching the extracted disease name, and then may control the disease related information output device 140 to output the detected disease related information.

To this end, the controller 120 includes a related disease name extraction module 121, a weighting module 122, a disease related information output module 123, and a disease information related advertisement module 124.

The related disease name extraction module 121 extracts the disease name based on the medical department and the drug among the prescription information input through the patient prescription interface 110.

In this connection, for example, hyperlipidemia, cardiovascular disease, gout, diabetes mellitus, osteoporosis, thyroid disease, kidney disease, glaucoma, benign prostatic hyperplasia, chronic obstructive pulmonary disease (COPD), pancreatitis, hepatitis, etc. may be predefined as primary disease names. In some cases, some thereof may be deleted from a list of the primary disease names or another disease may be added thereto.

Further, thrombosis, gastrointestinal disorder, sleep disorder, pain, etc. may be predefined as secondary disease names. A weight corresponding to the output priority or order may be allocated to the secondary disease names based on the severity of the disease. The weight may vary based on a situation.

When all of extracted disease names extracted by the related disease name extraction module 12 based on the medical department and the drugs belong to the primary disease names, the related disease name extraction module 12 may select all of the extracted disease names. When all of extracted disease names extracted by the related disease name extraction module 12 based on the medical department and the drugs do not belong to the primary disease names, that is, the extracted disease names belonging to the secondary disease name are extracted, the related disease name extraction module 12 may select at least one secondary disease name therefrom based on a predefined disease severity level thereof.

Further, when a single drug is used for different diseases and thus performs different functions, the related disease name extraction module 12 may extract the related disease name based on a combination of the single drug and a drug prescribed together therewith.

When the disease name is extracted by the related disease name extraction module 121, the weighting module 122 may assign weights to the disease related information to be provided to the patient. In this connection, the weights may correspond to the output priority or order of the disease related information. That is, the disease related information having the higher weight may be outputted at a higher output priority or order.

Specifically, the weights may be allocated such that the patient may first understand the basic information about the same disease most necessary for the patient, and then understand additional depth information about the same disease based on the understanding of the basic information. The more depth information may have the lower weight allocated thereto.

Thus, the higher weight may be allocated to the primary diseases, while the lower weight may be allocated to the secondary diseases. In this case, when a plurality of primary disease names are extracted, the weights may be allocated thereto based on the severity of the diseases. Further, when the secondary diseases are further extracted in addition to the primary diseases which is a serious disease, the weights may be allocated to secondary diseases based on the severity of the diseases.

Further, when first and second diseases have first and second severity levels respectively where the first level is higher than the second level, and three information about the first disease have first higher, medium and lower weights allocated thereto respectively, and three information about the second disease have second higher, medium and lower weights allocated thereto respectively, the second higher weight is higher than the first lower weight. Thus, a situation in which only disease information with high severity are first and collectively presented, and then only disease information with lower severity are collectively presented may be prevented.

The disease related information output module 123 may output the disease related information about the related disease name having the highest weight allocated thereto at a highest output priority or order. That is, the disease related information output module 123 may first output the disease related information about the related disease name having the highest weight. In other words, the disease related information output module 123 may output the disease related information about the related disease name having a corresponding weight allocated thereto at a corresponding output priority or order.

When the disease related information output module 123 outputs the disease related information, the disease information related advertisement module 124 may include the advertisements related to drugs, health supplements, and exercise equipment into the disease related information.

The storage 130 may include the disease related information database 131 and the disease information related advertisement database 132. The storage 130 may store the disease related information about the disease name extracted by the related disease name extraction module 121 into the disease related information database 131 under the control of the controller 120. The storage 130 may store advertisements related to the disease related information, such as advertisements related to the drugs, health supplements, and exercise equipment into the disease information related advertisement database 132.

The disease related information may include descriptions of the disease of the patient, lifestyle, diet, exercise therapy, nutrients as needed, and related general drugs for the patient.

The disease related information output device 140 may outputs the disease related information about the disease name extracted under the control of the controller 120. The disease related information output device 140 may include a display, a printer, and a speaker, etc. The advertisements for drugs, health supplements, and exercise equipment related to disease related information may be contained in the disease related information and thus may be outputted to the patient.

With reference to the accompanying drawings, a method for extracting a related disease from a patient prescription and providing extracted disease related information according to an embodiment of the present disclosure using the system having the above configuration will be described in detail as follows.

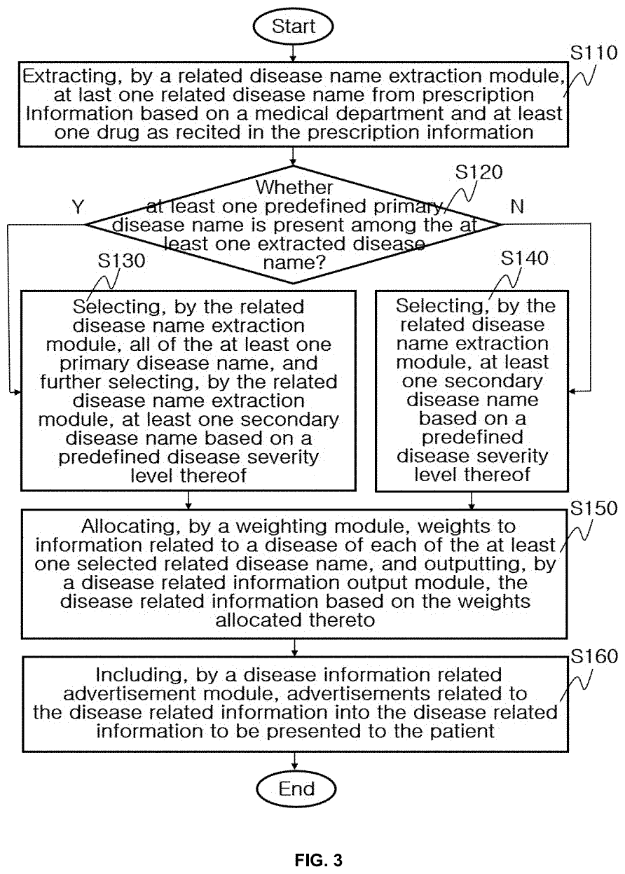

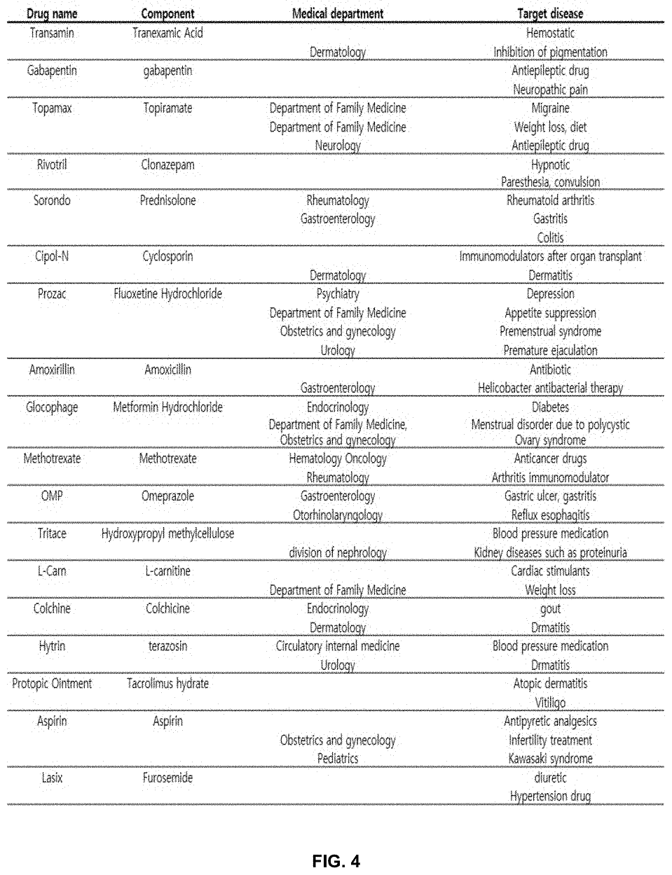

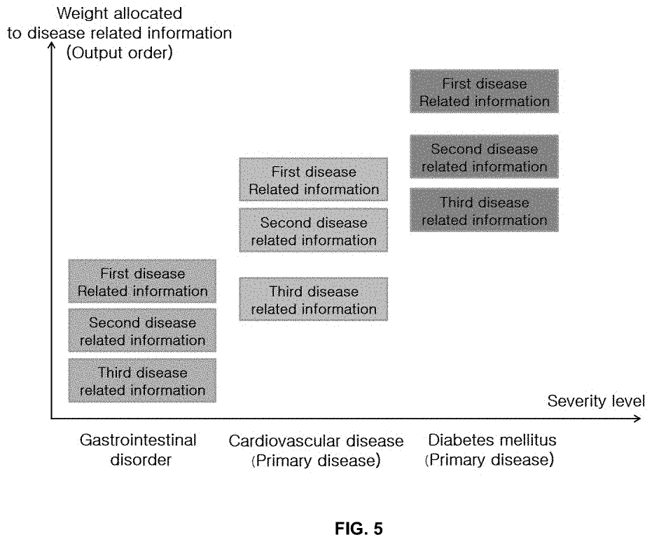

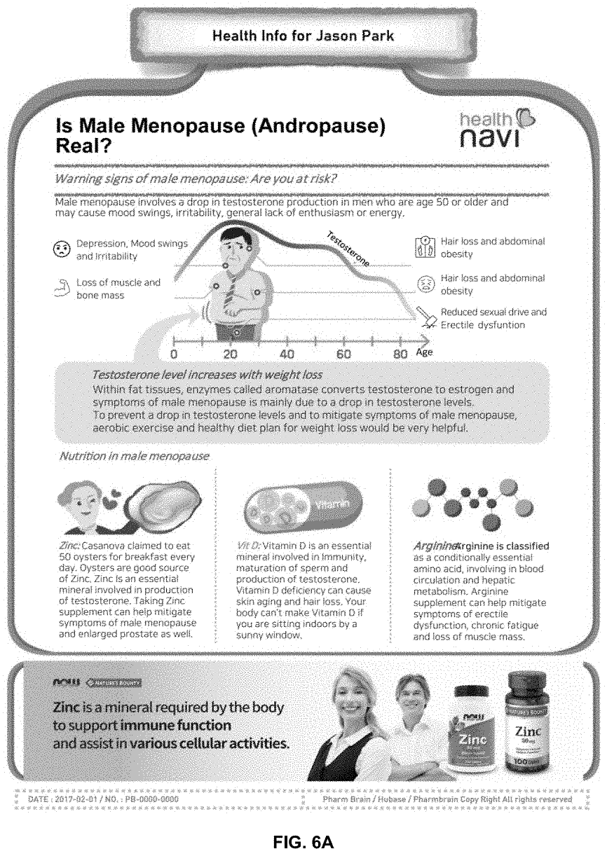

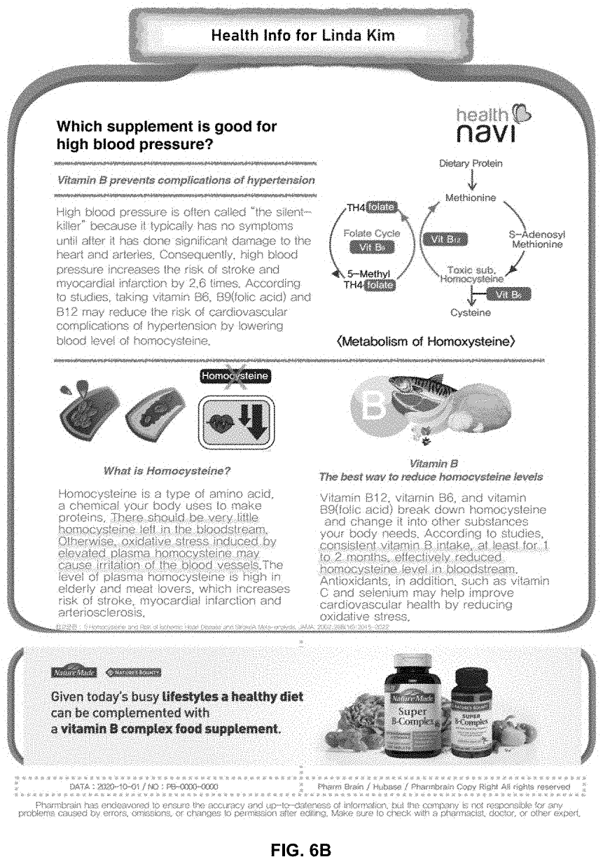

FIG. 3 is a flow chart of a method for extracting a related disease from a patient prescription and providing extracted disease related information according to an embodiment of the present disclosure. FIG. 4 is a diagram showing an example of extracting a related disease name based on a medical department in the method according to an embodiment of the present disclosure. FIG. 5 is a diagram showing a weight corresponding to an output priority or order of information about the extracted related disease based on a disease name and a disease severity in the method according to an embodiment of the present disclosure. FIG. 6A and FIG. 6B are images showing extracted disease related information and advertisements when a disease extracted from the patient prescription is diabetes mellitus according to an embodiment of the present disclosure.

Although not shown in the drawings, the user may use the patient prescription interface 110 to input the prescription information to the controller 120 of the computer. The prescription information may include prescribed drugs, classification of diseases, medical departments, whether medical insurance is applied, dosage, taking methods, etc. In some cases, the prescription information may be transmitted and inputted to the controller 120 of the computer from a hospital sever or computer through the Internet or via a wire or in a wireless manner.

Then, as shown in FIG. 3, the related disease name extraction module 121 of the controller 120 may receive the prescription information input via the patient prescription interface 110 and extract a related disease name from the prescription information based on the medical department and the drugs as recited in the prescription information (S110).

More specifically, the related disease name extraction module 121 of the controller 120 may search the disease related information database (DB) 131 for the disease related information matching the extracted disease name.

In this connection, the related disease name as extracted by the related disease name extraction module 121 of the controller 120 may refer to WHO standard ATC (Anatomical Therapeutic Chemical) codes. The Anatomical Therapeutic Chemical (ATC) Classification is an internationally accepted classification system for medicines that is maintained by the World Health Organization (WHO). The WHO assigns ATC codes to all active substances contained in medicines based on the therapeutic indication for the medicine. Using the ATC code, active substances are classified in groups at five different levels according to the organ or system on which they act and their therapeutic, pharmacological, and chemical properties. However, the present disclosure may not be limited thereto.

In this connection, when extracting the related disease name to the drug as prescribed, the same drug may be applied to different diseases based on different medical departments. This is shown in FIG. 4.

For example, the drug name ‘Prozac’ may be applied to ‘depression’ in a department of psychiatry, and may be applied to ‘appetite suppression’ in a family medicine department, and may be applied to ‘premenstrual syndrome’ in an obstetrics and gynecology department, and may be applied to ‘premature ejaculation’ in an urology department.

Further, a single drug may be used for different diseases and performs different functions. In this case, the related disease name extraction module 12 may extract the related disease name based on a combination of the single drug and a drug prescribed together therewith.

For example, ‘pheniramine’ may be used as antihistamines, for itchiness, and for rhinitis. When pheniramine is combined with ‘Sorondo’, the related disease name may be extracted as the itchy skin. When pheniramine is combined with ‘Cofrel’, the related disease name may be extracted as rhinitis or cold.

Then, the related disease name extraction module 121 of the controller 120 may be configured to determine whether a predefined primary disease name is present among the disease names as extracted in S110 (S120).

When it is determined in S120 that the predefined primary disease name is present among the disease names as extracted in S110, all of the extracted primary disease names may be selected.

In this connection, for example, hyperlipidemia, cardiovascular disease, gout, diabetes mellitus, osteoporosis, thyroid disease, kidney disease, glaucoma, benign prostatic hyperplasia, chronic obstructive pulmonary disease (COPD), pancreatitis, hepatitis, etc. may be predefined as the primary disease names. In some cases, some thereof may be deleted from a list of the primary disease names or another disease may be added thereto.

Further, when all of extracted disease names extracted by the related disease name extraction module 12 based on the medical department and the drugs do not belong to the primary disease names, that is, the extracted disease names belonging to the secondary disease name are extracted, the related disease name extraction module 12 may select at least one secondary disease name therefrom based on a predefined disease severity level thereof (S130).

In this connection, thrombosis, gastrointestinal disorder, sleep disorder, pain, etc. may be predefined as secondary disease names. A weight corresponding to the output priority or order may be allocated to the secondary disease names based on the severity of the disease. The weight may vary based on a situation.

An example in which all of the primary disease names are selected and then one or more secondary disease names other than the primary disease names are selected based on a predefined disease severity will be described as follows.

### Example 1. 68-Year-Old Male, Cardiology Department

| | | Whether extracted |
|---|---|---|
| | | disease is primary |
| Prescribed drug | Extracted disease | disease? |
| Astrix | Thrombosis | No |
| Lipitor 10 mg | Hyperlipidemia | Yes |
| Atacane 8 mg | Cardiovascular disease | Yes |
| Feburic | Gout | Yes |
| Diabex sustained-release | Diabetes mellitus | Yes |
| tablet 500 mg | | |
| H-2 | Gastrointestinal disorder | No |
| Stilnox CR | Sleep disorder | No |

Primary disease name: all of hyperlipidemia, cardiovascular disease, gout, diabetes mellitus

Secondary disease name: at least one among thrombosis, gastrointestinal disorder, and sleep disorder may be selected based on the disease severity thereof

That is, the four primary diseases: hyperlipidemia, cardiovascular disease, gout, and diabetes mellitus may be first selected and may be outputted in the highest order or priority. Then, remaining secondary diseases may be selected based on the disease severity thereof and may be outputted based on the disease severity thereof. In this case, thrombosis, sleep disorder and gastrointestinal disorder may be selected in this order or priority.

### Example 2. 52-Year-Old Male, Endocrinology Department

| | | Whether extracted |
|---|---|---|
| | | disease is primary |
| Prescribed drug | Extracted disease | disease? |
| Radix | Cardiovascular disease | Yes |
| Novask | Cardiovascular disease | Yes |
| Sorondo | Steroid | No |
| Zyroric | Gout | Yes |
| Tylenol | Anti-inflammatory | No |
| | analgesic drug | |
| OMP | Gastrointestinal disorder | No |

Primary disease name: both of cardiovascular disease and gout

Secondary disease name: one or more of steroid, anti-inflammatory analgesic drug, and gastrointestinal disorder may be selected based on the disease severity

In other words, the two primary diseases: cardiovascular disease, and gout may be first selected and may be outputted in the highest order or priority. Then, remaining secondary diseases may be selected based on the disease severity thereof and may be outputted based on the disease severity thereof. In this case, steroid, anti-inflammatory analgesic drug, and gastrointestinal disorder may be selected in this order or priority.

### Example 3. 59-Year Old Female, Cardiology Department

| | | Whether extracted |
|---|---|---|
| | | disease is primary |
| Prescribed drug | Extracted disease | disease? |
| Astrix | Thrombosis | No |
| Lipitor 10 mg | Hyperlipidemia | Yes |
| H-2 | Gastrointestinal disorder | No |
| Stilnox CR | Sleep disorder | No |

Primary disease name: hyperlipidemia

Secondary disease name: one or more of thrombosis, gastrointestinal disorder, and sleep disorder may be selected based on the disease severity

That is, the primary disease, that is, hyperlipidemia may be first selected and may be outputted in the highest order or priority. Then, remaining secondary diseases may be selected based on the disease severity thereof and may be outputted based on the disease severity thereof. In this case, thrombosis, gastrointestinal disorder, and sleep disorder may be selected in this order or priority.

Thus, the disease name may be extracted and selected based on the severity of the disease, while avoiding exclusion of the primary diseases.

When the disease related information output module 123 of the controller 120 has selected all of the predefined primary disease names in S130 and then has further selected the secondary disease names based on the disease severity level thereof, the disease related information output module 123 of the controller 120 may detect the disease related information matching the selected disease names from the disease related information database 131 of the storage 130, and output the detected disease related information based on the previously assigned weights thereto (S150).

In this connection, FIG. 5 is a diagram showing a weight corresponding to an output priority or order of the disease information about the extracted related disease based on the disease name and the disease severity. Referring to FIG. 5, an example of the process of assigning the weights to the disease related information when outputting the disease related information to be provided to the patient will be described.

First, first disease related information, second disease related information, and third disease related information may correspond to the basic information about the same disease most necessary to the patient, first additional depth information about the same disease, and second additional depth information about the same disease having deeper knowledge that that of the first additional depth information.

Further, the cardiovascular disease and the diabetes mellitus may be defined as the primary disease. The gastrointestinal disorder may be defined as the secondary disease. The primary disease may have a higher output priority or order than that of the secondary disease. When the number of the primary diseases is plural, that is, the primary diseases are the cardiovascular disease and the diabetes mellitus, the cardiovascular disease with the higher disease severity has the higher output priority or order allocated thereto than the diabetes mellitus has.

Further, When the number of the additionally selected secondary diseases other than the primary disease is plural, the additionally selected secondary diseases may have the output priority or order allocated thereto based on the disease severity level.

Moreover, when first and second diseases have first and second severity levels respectively where the first level is higher than the second level, and three information about the first disease have first higher, medium and lower weights allocated thereto respectively, and three information about the second disease have second higher, medium and lower weights allocated thereto respectively, the second higher weight is higher than the first lower weight. Thus, a situation in which only disease information with high severity are first and collectively presented, and then only disease information with lower severity are collectively presented may be prevented.

For example, the first disease related information of diabetes mellitus among the primary diseases may be output at the higher order than the third disease related information of the cardiovascular disease among the primary diseases may be. The first disease related information of the gastrointestinal disorder as the secondary disease may be output at the higher order than the third disease related information of the diabetes mellitus as the primary disease may be.

In this connection, in order to control the order of the output of the disease related information by the disease related information output module 123 of the controller 120, the weighting module 122 of the controller 120 may allocate the different weights to the basic information about the same disease most necessary to the patient, first additional depth information about the same disease, and second additional depth information about the same disease having deeper knowledge that that of the first additional depth information, respectively. Further, the weighting module 122 of the controller 120 may allocate the different weights to the primary disease and the secondary disease. Further, the weighting module 122 of the controller 120 may allocate the different weights to primary diseases based on the disease severity level. Further, the weighting module 122 of the controller 120 may allocate the different weights to the secondary diseases based on the disease severity level.

Thus, the disease related information output module 123 of the controller 120 may detect the disease related information matching the disease names selected based on the weights allocated thereto by the weighting module 122 from the disease related information database 131 of the storage 130, and output the detected disease related information based on the previously assigned weights thereto (S150).

In this case, the output may be implemented by the disease related information output device 140 in visual and/or audible manner. For example, the disease related information output device 140 may output the information in a text and/or graphic form to the patient.

When the extracted disease is a specific disease occurring in unusual circumstances such as Corona 19 epidemic, the highest priority weight may be allocated thereto such that information about the specific disease may be output at the highest output order.

Further, when it is difficult to extract the disease name because it is difficult to identify the medical department, as in a local clinic, or when the disease related information to be output is exhausted or the extracted disease does not match the prepared disease information, the controller 120 may be configured such that health information related to each medical department may be provided to the patient, or general health knowledge information may be provided to the patient.

In addition, the controller 120 may be configured such that prior to the provision of the related disease information about the disease as extracted based on the drug, the side effects of the drug, the dosage method thereof, and precautions when taking the drug are described in more detail.

Further, the controller 120 may be configured such that when presenting the disease related information, advertisements related to the disease related information may be contained in a lower portion or an appropriate position of a paper and presented to patients who have been provided with the disease related information. Thus, pharmaceutical companies or wholesale and retailers in need of drug-related promotion marketing may do effective marketing.

To this end, the disease information related advertisement module 124 of the controller 120 may search the disease information related advertisement database 132 of the storage 130 for the disease information related advertisement matching the disease related information to be output, and may include the disease information related advertisement into the disease related information, which in turn may be provided to the disease related information output device 140 (S160). The disease related information output device 140 may visually and/or audibly present the disease related information containing therein the disease information related advertisement.

In one example, when it is determined that there is no predefined primary disease name among the extracted disease names in step S120, the secondary disease name may be additionally selected based on the predefined disease severity level (S140). Then, the disease related information about the secondary disease name may be output through S150, and S160.

FIG. 6A and FIG. 6B are images showing extracted disease related information and advertisements when a disease extracted from the patient prescription is diabetes mellitus according to an embodiment of the present disclosure. In this example, the disease related information matching the disease name as extracted may be related to the male menopause and high blood pressure as shown in FIG. 6A and FIG. 6B.

The method for extracting the related disease from the patient prescription and providing the extracted disease related information according to one embodiment of the inventive concept as described above may be implemented using a program (or application) to be executed in combination with a computer as hardware and stored in a medium.

The program may include codes coded in computer languages such as C, C++, JAVA, and machine language that a processor (CPU) of the computer may read through a device interface thereof, in order for the computer to read the program and execute methods implemented using the program. The code may include a functional code related to a function defining functions required to execute the methods, and an execution procedure-related control code necessary for the processor of the computer to execute the functions in a predetermined procedure. Moreover, the code may further include a memory reference-related code indicating a location (address) of an internal memory of the computer or an external memory thereto in which additional information or media necessary for the processor to execute the functions is stored. Moreover, when the processor of the computer needs to communicate with any other remote computer or server to execute the functions, the code may further include a communication-related code indicating how to communicate with any other remote computer or server using a communication module of the computer, and indicating information or media to be transmitted and received during the communication.

The storage medium means a medium that stores data semi-permanently, rather than a medium for storing data for a short moment, such as a register, a cache, or a memory, and that may be readable by a machine. Specifically, examples of the storage medium may include, but may not be limited to, ROM, RAM, CD-ROM, a magnetic tape, a floppy disk, and an optical data storage device. That is, the program may be stored in various recording media on various servers to which the computer may access or on various recording media on the user's computer. Moreover, the medium may be distributed over a networked computer system so that a computer readable code may be stored in a distributed scheme.

The steps of the method or the algorithm described in connection with the embodiments of the inventive concept may be implemented directly in hardware, a software module executed by hardware, or a combination thereof. The software modules may reside in random access memory (RAM), read only memory (ROM), erasable programmable ROM (EPROM), electrically erasable programmable ROM (EEPROM), a flash memory, a hard disk, a removable disk, CD-ROM, or any form of a computer readable recording medium well known in the art.

According to the method for extracting the related disease from the patient prescription and providing the extracted disease related information according to one embodiment of the inventive concept, while prioritizing the predefined primary disease name based on the medical department and the prescribed drug on the patient prescription, one or more secondary disease names may be additionally extracted based on the predefined severity level thereof. In this connection, the disease related information may be outputted based on the weights allocated based on whether the extracted disease is the primary disease and based on the disease severity level thereof. Thus, when extracting the related disease based on the drug information on the patient prescription, the primary disease may be prevented from being excluded from the output. Further, the disease-related information may be output based on the predefined diseases severity level, thereby facilitating the disease healing, preventing recurrence thereof, and maintaining health of the patient continuously.

Further, according to the method for extracting the related disease from the patient prescription and providing the extracted disease related information according to one embodiment of the inventive concept, the weighting module performs weighting such that when first and second diseases have first and second severity levels respectively where the first level is higher than the second level, and three information about the first disease have first higher, medium and lower weights allocated thereto respectively, and three information about the second disease have second higher, medium and lower weights allocated thereto respectively, the second higher weight is higher than the first lower weight. Thus, a situation in which only disease information with high severity are first and collectively presented, and then only disease information with lower severity are collectively presented may be prevented.

Furthermore, according to the method for extracting the related disease from the patient prescription and providing the extracted disease related information according to one embodiment of the inventive concept, the disease information related advertisement module may include the advertisements related to the disease related information into the disease related information to be presented to the patient. Thus, pharmaceutical companies or wholesale and retailers in need of drug-related promotion marketing may do effective marketing.

The systems, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “determine,” “identify,” and “provide” to describe the disclosed methods. These terms are high-level abstractions of the actual operations that are performed. The actual operations that correspond to these terms will vary depending on the particular implementation and are readily discernible by one of ordinary skill in the art.

What is claimed is:

1. A method for extracting a related disease from a patient prescription and providing extracted disease related information using a system,
wherein the system includes a patient prescription interface, a controller, a storage, and a disease related information output device,
wherein the controller includes a related disease name extraction module, a weighting module, and a disease related information output module,
wherein the method comprises:
receiving, by the related disease name extraction module, prescription information via the patient prescription interface;
extracting, by the related disease name extraction module, at last one related disease name from the prescription information based on a medical department and at least one drug as recited in the prescription information;
determining, by the related disease name extraction module, whether at least one predefined primary disease name is present among the at least one extracted disease name;
upon determination that the at least one predefined primary disease name is present among the at least one extracted disease name, selecting, by the related disease name extraction module, all of the at least one primary disease name, and further selecting, by the related disease name extraction module, at least one predefined secondary disease name based on a predefined disease severity level thereof;
upon determination that the at least one predefined primary disease name is not present among the at least one extracted disease name, selecting, by the related disease name extraction module, at least one predefined secondary disease name based on a predefined disease severity level thereof;
allocating, by the weighting module, weights to information related to a disease of each of the at least one selected related disease name, wherein the weights correspond to output orders or priorities of the disease related information when the disease related information output module outputs the disease related information; and
outputting, by the disease related information output module, the disease related information based on the weights allocated thereto,
wherein the weighting module allocates first to third weights to basic information about the same disease most necessary to a patient, first additional depth information about the same disease, and second additional depth information about the same disease having deeper knowledge that that of the first additional depth information, respectively, wherein the first to third weights decrease in this order,
wherein the weighting module allocates weights to information about the primary disease and information about the secondary disease, respectively, wherein the weight allocated to the information about the primary disease is higher than the weight allocated to the information about the secondary disease,
wherein the weighting module allocates different weights to about information the primary diseases based on disease severity levels thereof, or allocates different weights to information about the secondary diseases based on disease severity levels thereof, wherein a higher weight corresponds to a higher severity level.

2. The method of claim 1, wherein the weighting module performs weighting such that: when first and second diseases have first and second severity levels respectively where the first level is higher than the second level, and three information about the first disease have first higher, medium and lower weights allocated thereto respectively, and three information about the second disease have second higher, medium and lower weights allocated thereto respectively,
the second higher weight is higher than the first lower weight.

3. The method of claim 1, wherein the controller further includes a disease information related advertisement module, wherein the method further comprises including, by the disease information related advertisement module, advertisements related to the disease related information into the disease related information to be presented to the patient.
